SEQUENCE LISTING (25 sequences)

SEQ ID NO: 1 - DNA, 99 bp, Solanum galapagense
  source 1..99: mol_type = genomic DNA
tgtagaagattgatttatctgctgccactctcaaagctaaggcctggtgtttatctggaccagcagtgttctcgaaaccaatgtccttagcaacgaaat

SEQ ID NO: 2 - DNA, 20 bp, synthetic construct
  misc_feature 1..20: note = Forward primer SNP 1
  source 1..20: mol_type = other DNA
gctgccactctcaaagctaa

SEQ ID NO: 3 - DNA, 17 bp, synthetic construct
  misc_feature 1..17: note = Probe for R allele
  source 1..17: mol_type = other DNA
cagataaacaccaggcc

SEQ ID NO: 4 - DNA, 17 bp, synthetic construct
  misc_feature 1..17: note = Probe for S allele
  source 1..17: mol_type = other DNA
cagataaacatcaggcc

SEQ ID NO: 5 - DNA, 22 bp, synthetic construct
  misc_feature 1..22: note = Reverse primer SNP 1
  source 1..22: mol_type = other DNA
ggtttcgagaacactgctggtc

SEQ ID NO: 6 - DNA, 135 bp, Solanum galapagense
  variation 6: note = n is a, c, g, or t
  variation 17: note = n is a, c, g, or t
  variation 39..45: note = n is a, c, g, or t
  variation 53: note = n is a, c, g, or t
  variation 116: note = n is a, c, g, or t
  source 1..135: mol_type = genomic DNA
gctcgnttaaggatggngatgtgaagatggaggaggaannnnnnntaaagagnaggagggaaagtgaaaagccaccagaggatgactgttgtcctatttgctttggggatttctcnattccttgtaaaaccaact

SEQ ID NO: 7 - DNA, 18 bp, synthetic construct
  misc_feature 1..18: note = Forward primer SNP 2
  source 1..18: mol_type = other DNA
gtgaagatggaggaggaa

SEQ ID NO: 8 - DNA, 18 bp, synthetic construct
  misc_feature 1..18: note = Probe for R allele
  source 1..18: mol_type = other DNA
aacagtcatcctctggtg

SEQ ID NO: 9 - DNA, 19 bp, synthetic construct
  misc_feature 1..19: note = Probe for S allele
  source 1..19: mol_type = other DNA
ataggacaacagtcatccc

SEQ ID NO: 10 - DNA, 18 bp, synthetic construct
  misc_feature 1..18: note = Reverse primer SNP 2
  source 1..18: mol_type = other DNA
gagaaatccccaaagcaa

SEQ ID NO: 11 - DNA, 110 bp, Solanum galapagense
  source 1..110: mol_type = genomic DNA
aataggagcgcaagacgtcggtactgattagcgataagttcagaatcatgggtttggttggaaggaatttgaagaatagagtaataatcaagatgctggttgttgatccg

SEQ ID NO: 12 - DNA, 18 bp, synthetic construct
  misc_feature 1..18: note = Forward primer SNP 3
  source 1..18: mol_type = other DNA
acgtcggtactgattagc

SEQ ID NO: 13 - DNA, 15 bp, synthetic construct
  misc_feature 1..15: note = Probe for R allele
  source 1..15: mol_type = other DNA
ccaaccaaacccatg

SEQ ID NO: 14 - DNA, 16 bp, synthetic construct
  misc_feature 1..16: note = probe for S allele
  source 1..16: mol_type = other DNA
tccaaccaaactcatg

SEQ ID NO: 15 - DNA, 22 bp, synthetic construct
  misc_feature 1..22: note = Reverse primer SNP 3
  source 1..22: mol_type = other DNA
ccagcatcttgattattactct

SEQ ID NO: 16 - DNA, 212 bp, Solanum galapagense
  source 1..212: mol_type = genomic DNA
tgccaatgtttaggctctcagcctagacagatgactgatgagaatgattactttaaaatcaattatccaaattcataaagtagataatggttttttaacctttttggttattttatcaacttcgtctatgtagacaacccattgccgaactgcttctacattgaagttagcagcctgtaaacaaacatgaacaccaaaaatttataattgaa

SEQ ID NO: 17 - DNA, 24 bp, synthetic construct
  misc_feature 1..24: note = Forward primer SNP 4
  source 1..24: mol_type = other DNA
gcctagacagatgactgatgagaa

SEQ ID NO: 18 - DNA, 20 bp, synthetic construct
  misc_feature 1..20: note = Probe for R allele
  source 1..20: mol_type = other DNA
tgtagaagcagtttggcaat

SEQ ID NO: 19 - DNA, 18 bp, synthetic construct
  misc_feature 1..18: note = Probe for S allele
  source 1..18: mol_type = other DNA
agaagcagttcggcaatg

SEQ ID NO: 20 - DNA, 25 bp, synthetic construct
  misc_feature 1..25: note = Reverse primer SNP 4
  source 1..25: mol_type = other DNA
gttcatgtttgtttacaggctgcta

SEQ ID NO: 21 - DNA, 118 bp, Solanum galapagense
  variation 7: note = n is a, c, g, or t
  variation 10: note = n is a, c, g, or t
  variation 13: note = n is a, c, g, or t
  variation 40: note = n is a, c, g, or t
  variation 43: note = n is a, c, g, or t
  variation 49: note = n is a, c, g, or t
  variation 54: note = n is a, c, g, or t
  variation 57..58: note = n is a, c, g, or t
  variation 73: note = n is a, c, g, or t
  variation 97: note = n is a, c, g, or t
  source 1..118: mol_type = genomic DNA
gccgccnccnccntcacgtcggccacctccgtagccgccnccnccaccntagcntcnnccaccgccttcacgncggccacctccgtaaccaccaccncctcttccacctccacctcca

SEQ ID NO: 22 - DNA, 16 bp, synthetic construct
  misc_feature 1..16: note = Forward primer SNP 5
  source 1..16: mol_type = other DNA
ggccacctccgtagcc

SEQ ID NO: 23 - DNA, 16 bp, synthetic construct
  misc_feature 1..16: note = Probe for R allele
  source 1..16: mol_type = other DNA
ctccgtaaccaccacc

SEQ ID NO: 24 - DNA, 14 bp, synthetic construct
  misc_feature 1..14: note = Probe for S allele
  source 1..14: mol_type = other DNA
ccgtaaccaccccc

SEQ ID NO: 25 - DNA, 20 bp, synthetic construct
  misc_feature 1..20: note = Reverse primer SNP 5
  source 1..20: mol_type = other DNA
tggaggtggaggtggaagag

(19) United States
(12) Patent Application Publication
(10) Pub. No.: US 2025/0143244 A1
(43) Pub. Date: May 8, 2025

(54) NOVEL TOMATO PLANTS WITH TOBRFV RESISTANCE

(71) Applicant: SYNGENTA CROP PROTECTION AG, Basel (CH)

(72) Inventors: Celine Labourey, Sarrians (FR); Elise Albert, Saint-Sauveur (FR); Arnaud Chassaigne, Saint-Sauveur (FR)

(73) Assignee: SYNGENTA CROP PROTECTION AG, Basel (CH)

(21) Appl. No.: 18/837,632

(22) PCT Filed: Feb. 16, 2023
(86) PCT No.: PCT/EP2023/053979

§ 371 Date: Aug. 12, 2024

(30) Foreign Application Priority Data
Feb. 17, 2022 (EP) 22157283.7

(51) Int. Cl. A01H 1/00; A01H 5/08; A01H 6/82

(52) U.S. Cl. CPC A01H 1/126; A01H 5/08; A01H 6/825

(57) ABSTRACT

The present invention relates to novel tomato plants displaying an increased resistance to ToBRFV infection. The present invention also relates to seeds and parts of said plants, for example fruits. The present invention further relates to methods of making and using such seeds and plants. The present invention also relates to novel genetic sequences associated with said increased resistance and to molecular markers associated with said novel genetic sequences.

### FIELD OF THE INVENTION

The present invention relates to novel tomato plants displaying an increased resistance to ToBRFV infection. The present invention also relates to seeds and parts of said plants, for example fruits. The present invention further relates to methods of making and using such seeds and plants. The present invention also relates to novel genetic sequences associated with said increased resistance and to molecular markers associated with said novel genetic sequences.

### BACKGROUND OF THE INVENTION

Viruses, such as those belonging to the Tobamovirus genus, cause considerable damage to tomato growers, irrespective of the tomato types and cultivation methods. Infections by Tobamoviruses (e.g., by Tobacco Mosaic Virus (TMV), Tomato Mosaic Virus (ToMV) or Tomato Mild Mottle Virus (ToMMV)) typically spread rapidly across fields, either via mechanical transmission through field workers, tools and manual handling of plants and fruits, or via natural vectors such as insects, which are equally challenging to control.

In 2014 and 2015, a new Tobamovirus was first reported on tomatoes in Israel and Jordan respectively (Luria et al., 2017, Salem et al., 2016). It was named Tomato Brown Rugose Fruit Virus (ToBRFV) after one of the main symptoms caused to the crop, namely the deformation of fruits with yellow and/or brown areas. This ToBRFV outbreak is a particular concern for the tomato industry because the virus was shown to be able to overcome the tobamovirus resistance gene Tm-2². There is therefore a need for solutions for the grower and the seed industry to be able to grow ToBRFV resistant tomato crops.

Possible sources for ToBRFV resistance have been described in the patent and non-patent literature.

For instance, WO 2018/219941 reports QTLs located on chromosomes 6, 9 and 11, while WO 2019/110821, WO 2020/018783, WO 2021/110855 and WO 2021/170868 also disclose QTLs or genes located on chromosome 11.

WO 2020/148021 and WO 2022/013452 report a QTL or gene located on chromosome 8.

Finally, WO 2021/213892 discloses an alternative allele of a known resistance gene, tm-1, located on chromosome 2.

Despite the number of described loci, it is yet unclear what (combination of) QTLs or genes will effectively provide resistance to ToBRFV in the fields because the mechanisms underlying the ToBRFV infection have yet to be determined. For instance, Zinger et al. suggest that a locus located on chromosome 2 interacts with a locus located on chromosome 11 to control ToBRFV resistance (Zinger et al., 2021).

Consequently, there is a need for alternative solutions to further improve ToBRFV control in plants, especially in tomato plants.

### SUMMARY OF THE INVENTION

The present invention addresses the need for an improved resistance to ToBRFV by providing novel tomato plants comprising an increased ToBRFV resistance trait. By identifying one novel QTL associated with increased ToBRFV resistance in a breeding population and by introgressing its corresponding sequence into elite tomato plants, the ToBRFV resistance capability of the tomato plant was increased, which has a positive impact on overall plant performance. The ToBRFV resistance QTL and its corresponding introgressed sequence, located on chromosome 1 (QTL1), is of a recessive nature, hence two copies of the sequence are necessary to provide the improved ToBRFV resistance phenotype.

Altogether, the characteristics of the improved ToBRFV resistant tomato plant disclosed within the present invention provide a tomato grower with a novel solution to enhance economic and commercial efficiency when deploying tomato varieties in a ToBRFV pressured field.

In a first embodiment, the invention provides a cultivated tomato plant, preferably a cultivated Solanum lycopersicum plant resistant to ToBRFV infection, comprising in its genome an introgressed sequence from a Solanum galapagense plant which confers resistance to ToBRFV, wherein said introgressed sequence is located on chromosome 1 and comprises at least one of the following SNP markers:
a) an A genotype in the homozygous state for SNP marker 2 at a position corresponding to position 79 in SEQ ID NO: 6;
b) a G genotype in the homozygous state for SNP marker 3 at a position corresponding to position 51 in SEQ ID NO: 11; and/or
c) a G genotype in the homozygous state for SNP marker 4 at a position corresponding to position 147 in SEQ ID NO: 16.

Further, the plant of the previous embodiment wherein:
a) the A genotype for SNP marker 2 can be identified in a PCR by amplification of a nucleic acid fragment with a pair of oligonucleotide primers: forward primer of SEQ ID NO: 7 and reverse primer of SEQ ID NO: 10, and probe of SEQ ID NO: 8;
b) the G genotype for SNP marker 3 can be identified in a PCR by amplification of a nucleic acid fragment with a pair of oligonucleotide primers: forward primer of SEQ ID NO: 12 and reverse primer of SEQ ID NO: 15, and probe of SEQ ID NO: 13; and/or
c) the G genotype for SNP marker 4 can be identified in a PCR by amplification of a nucleic acid fragment with a pair of oligonucleotide primers: forward primer of SEQ ID NO: 17 and reverse primer of SEQ ID NO: 20, and probe of SEQ ID NO: 18.

In a further embodiment of the invention, said ToBRFV resistance-conferring introgressed sequence comprises at least one of SEQ ID NO: 6, SEQ ID NO: 11, and/or SEQ ID NO: 16, or a sequence that is at least 80%, preferably at least 85%, more preferably at least 90%, even more preferably at least 95% identical to one or more of said sequences.

In a further embodiment of the invention, said introgressed sequence is comprised in tomato plant 19TEP080670, representative seed of which is deposited under NCIMB Accession No. 43938, or a progeny or an ancestor thereof.

In a further embodiment, the invention provides a plant according to any of the preceding embodiments wherein said plant is obtained by crossing tomato plant 19TEP080670, representative seed of which is deposited under NCIMB Accession No. 43938, or a progeny or an ancestor thereof, with a tomato plant that does not contain said ToBRFV resistance-conferring introgressed sequence.

In a further embodiment, the invention provides a plant according to any of the preceding embodiments, wherein said plant is an inbred, a dihaploid, a diploid, or a hybrid plant.

It is a further embodiment to provide a plant part, organ or tissue obtainable from a tomato plant according to any of preceding embodiments, including but not limiting to leaves, stems, roots, flowers or flower parts, fruits, shoots, gametophytes, sporophytes, pollen, anthers, microspores, egg cells, zygotes, embryos, meristematic regions, callus tissue, seeds, cuttings, cell or tissue cultures or any other part or product of the plant which still exhibits the ToBRFV resistance trait according to the invention, particularly when grown into a plant that produces fruits.

In a further embodiment, the invention provides a seed that produces a plant according to any of the preceding embodiments.

In a further embodiment, the invention provides a method for producing a cultivated tomato plant, preferably a cultivated Solanum lycopersicum plant resistant to ToBRFV infection comprising the steps of
a) crossing a plant according to any one of the preceding embodiments with a cultivated tomato plant lacking said ToBRFV resistance-conferring introgressed sequence;
b) selecting a progeny plant comprising said introgressed sequence located on chromosome 1 conferring resistance to ToBRFV, said selecting step comprising detecting at least one of the following SNP markers:
   i) an A genotype in the homozygous state for SNP marker 2 at a position corresponding to position 79 in SEQ ID NO: 6;
   ii) a G genotype in the homozygous state for SNP marker 3 at a position corresponding to position 51 in SEQ ID NO: 11; and/or
   iii) a G genotype in the homozygous state for SNP marker 4 at a position corresponding to position 147 in SEQ ID NO: 16;
      thereby producing a plant with enhanced resistance to ToBRFV.

In a further embodiment, the invention relates to the method of any of the preceding embodiments, wherein the method further comprises:
c) selfing the selected progeny or crossing the selected progeny with another tomato plant to produce further progeny.

In a further embodiment, the invention relates to the method of the preceding embodiment, wherein further progeny is selected and selfed/crossed for 2 to 10 more generations. In a further embodiment, the invention relates to the method of any of the preceding embodiments, wherein the plant of step a) is tomato plant 19TEP080670, representative seed of which is deposited under NCIMB Accession No. 43938, or a progeny or an ancestor thereof.

In a further embodiment, the invention relates to a method for producing a F1 tomato plant exhibiting resistance to ToBRFV, the method comprising crossing an inbred tomato plant, which is a plant according to any one of the preceding embodiments, with a different inbred tomato plant to produce F1 hybrid progeny.

In a further embodiment, the invention provides a method for identifying a cultivated tomato plant, preferably a cultivated Solanum lycopersicum plant, exhibiting resistance to ToBRFV and having at least one copy of said ToBRFV resistance-conferring introgressed sequence, said method comprising the step of detecting at least one of the following SNP markers:
a) an A genotype in the homozygous state for SNP marker 2 at a position corresponding to position 79 in SEQ ID NO: 6;
b) a G genotype in the homozygous state for SNP marker 3 at a position corresponding to position 51 in SEQ ID NO: 11; and/or
c) a G genotype in the homozygous state for SNP marker 4 at a position corresponding to position 147 in SEQ ID NO: 16;
   thereby identifying a tomato plant exhibiting resistance to ToBRFV.

In a further embodiment, the invention relates to the method of the preceding embodiment, wherein said method further comprises selecting a tomato plant comprising said at least one or more SNP markers and crossing the selected tomato plant with a second tomato plant to produce progeny tomato plants that comprise at least one of said SNP markers and exhibits increased resistance to ToBRFV.

### BRIEF DESCRIPTION OF THE DRAWINGS

FIG. 1 shows ToBRFV pathology assay pictures representative of the disease scale used and described in Example 2C. (a) Rating Susceptible (S or 1); (b) Rating Intermediate Resistance (IR or 5); (c) Rating High Resistance (HR or 9).

FIG. 2 shows the results of a ToBRFV pathology assay carried out with a susceptible check (a) and the donor LA0483 plant (b).

FIG. 3 shows the results of a Tobamovirus pathology assay carried out with the donor LA0483 plant inoculated with pathotype 0 (a), ToMV pathotype 2 (b) and without virus (mock trial) (c).

### DETAILED DESCRIPTION OF THE INVENTION

### Definitions

The technical terms and expressions used within the scope of this application are generally to be given the meaning commonly applied to them in the pertinent art of plant breeding and cultivation if not otherwise indicated herein below.

As used in this specification and the appended claims, the singular forms “a”, “an”, and “the” include plural referents unless the context clearly dictates otherwise. Thus, for example, reference to “a plant” includes one or more plants, and reference to “a cell” includes mixtures of cells, tissues, and the like.

A “cultivated tomato” or an “elite tomato” plant is understood within the scope of the invention to refer to a plant that is no longer in the natural state but has been developed and domesticated by human care and for agricultural use and/or human consumption, and excludes wild tomato accessions, such as Solanum galapagense accessions. As a matter of example, in embodiments, a cultivated or elite tomato plant according to the present invention is capable of growing edible fruits. Alternatively, or additionally, the cultivated tomato plant is a hybrid plant. Alternatively, or additionally, the cultivated tomato plant is a cultivated Solanum lycopersicum plant.

An “allele” is understood within the scope of the invention to refer to alternative or variant forms of various genetic units identical or associated with different forms of a gene or of any kind of identifiable genetic determinant such as a QTL, which are alternative in inheritance because they are situated at the same locus in homologous chromosomes. Such alternative or variant forms may be the result of single nucleotide polymorphisms, insertions, inversions, translocations or deletions, or the consequence of gene regulation caused by, for example, chemical or structural modification, transcription regulation or post-translational modification/regulation. In a diploid cell or organism, the two alleles of a given gene or genetic element typically occupy corresponding loci on a pair of homologous chromosomes.

Relatively speaking, the term “improved ToBRFV resistance” or “increased ToBRFV resistance” is herein understood to mean that a plant according to the present invention, e.g., comprising an introgressed sequence from a Solanum galapagense plant which confers resistance to ToBRFV, wherein said introgressed sequence is located on chromosome 1 and comprises at least one of SNP markers 2 to 4, is more resistant to ToBRFV when compared with a plant lacking said introgressed sequence.

“Improved ToBRFV resistance” is understood within the scope of the invention to mean a tomato plant which has a statistically significant improved resistance to ToBRFV compared to a control tomato plant lacking the introgressed sequence of the invention (for example as described in the Example section), using standard error and/or at P<0.05 or P<0.01 using Student's test and/or a Lod score>3.

“Phenotype” is understood within the scope of the invention to refer to a distinguishable characteristic(s) of a genetically controlled trait.

A “control tomato plant” is understood within the scope of the invention to mean a tomato plant that has substantially the same genetic background as the cultivated tomato plant of the present invention wherein the control plant does not have the introgressed sequence of the present invention linked to improved ToBRFV resistance. A control tomato plant can be a tomato plant belonging to the same plant variety and does not comprise the introgressed sequence of the present invention. The control tomato plant is grown for the same length of time and under the same conditions as the cultivated tomato plant of the present invention. Plant variety is herein understood according to definition of UPOV. Thus, an ideal control tomato plant may be a near-isogenic line, an inbred line or a hybrid provided that they have the same genetic background as the tomato plant of the present invention except the control plant does not have the introgressed sequence of the present invention linked to improved ToBRFV resistance.

The term “trait” refers to a characteristic or a phenotype. In the context of the present invention, a ToBRFV resistance trait is an improved ToBRFV resistance trait. A trait may be inherited in a dominant or recessive manner, or in a partial, semi-or incomplete-dominant manner. In the context of the present invention, the ToBRFV resistance-conferring introgressed sequence located on chromosome 1 is recessive. A tomato plant of the invention is therefore homozygous for the trait. Furthermore, a trait may be monogenic or polygenic, or may result from the interaction of one or more genes with the environment. In the context of the present invention, the ToBRFV resistance-conferring introgressed sequence located on chromosome 1 is sufficient to confer, alone, the improved ToBRFV resistance trait, which is therefore monogenic.

The terms “hybrid”, “hybrid plant”, and “hybrid progeny” refer to an individual produced from genetically different parents (e.g., a genetically heterozygous or mostly heterozygous individual).

The term “inbred line” refers to a genetically homozygous or nearly homozygous population. An inbred line, for example, can be derived through several cycles of brother/sister breeding or of selfing or in dihaploid production.

The term “dihaploid line” refers to stable inbred lines issued from anther culture. Some pollen grains (haploid) cultivated on specific medium and conditions can develop plantlets containing n chromosomes. These plantlets are then “doubled” and contain 2n chromosomes. The progeny of these plantlets is named “dihaploid” and are essentially no longer segregating (stable).

The term “genetically fixed” refers to a genetic sequence which has been stably incorporated into the genome of a plant that normally does not contain said genetic sequence. When genetically fixed, the genetic sequence can be transmitted in an easy and predictable manner to other plants by sexual crosses.

The term “rootstock” refers to a plant used as a receptacle for a scion plant. Typically, the rootstock plant and the scion plant are of different genotypes. In embodiments, plants according to the present invention are used as rootstock plants.

The term “plant” or “plant part” refers hereinafter to a plant part, organ or tissue obtainable from a tomato plant according to the invention, including but not limiting to leaves, stems, roots, flowers or flower parts, fruits, shoots, gametophytes, sporophytes, pollen, anthers, microspores, egg cells, zygotes, embryos, meristematic regions, callus tissue, seeds, cuttings, cell or tissue cultures or any other part or product of the plant which still exhibits the improved ToBRFV resistance trait according to the invention, particularly when grown into a plant that produces fruits.

A “plant” is any plant at any stage of development.

A tomato plant seed is a seed which grows into a tomato plant according to any of the embodiments.

A “plant cell” is a structural and physiological unit of a plant, comprising a protoplast and a cell wall. The plant cell may be in form of an isolated single cell or a cultured cell, or as a part of higher organized unit such as, for example, plant tissue, a plant organ, or a whole plant.

“Plant cell culture” means cultures of plant units such as, for example, protoplasts, cell culture cells, cells in plant tissues, pollen, pollen tubes, ovules, embryo sacs, zygotes and embryos at various stages of development.

A “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units.

As used herein, the term “breeding”, and grammatical variants thereof, refer to any process that generates a progeny individual. Breeding can be sexual or asexual, or any combination thereof. Exemplary non-limiting types of breeding include crossings, selfing, doubled haploid derivative generation, and combinations thereof.

As used herein, the phrase “established breeding population” refers to a collection of potential breeding partners produced by and/or used as parents in a breeding program, e.g., a commercial breeding program. The members of the established breeding population are typically well-characterized genetically and/or phenotypically. For example, several phenotypic traits of interest might have been evaluated, e.g., under different environmental conditions, at multiple locations, and/or at different times. Alternatively, or in addition, one or more genetic loci associated with expression of the phenotypic traits might have been identified and one or more of the members of the breeding population might have been genotyped with respect to the one or more genetic loci as well as with respect to one or more genetic markers that are associated with the one or more genetic loci.

As used herein, the phrase “diploid individual” refers to an individual that has two sets of chromosomes, typically one from each of its two parents. However, it is understood that in some embodiments a diploid individual can receive its “maternal” and “paternal” sets of chromosomes from the same single organism, such as when a plant is selfed to produce a subsequent generation of plants.

“Homozygous” is understood within the scope of the invention to refer to like alleles at one or more corresponding loci on homologous chromosomes. In the context of the invention, a tomato plant comprising two identical copies of a particular introgressed sequence at a particular locus, e.g., the introgressed sequence located on chromosome 1, is homozygous on the corresponding locus.

“Heterozygous” is understood within the scope of the invention to refer to unlike alleles at one or more corresponding loci on homologous chromosomes.

A “dominant” allele is understood within the scope of the invention to refer to an allele which determines the phenotype when present in the heterozygous or homozygous state.

A “semi-dominant” allele is understood within the scope of the invention to refer to an allele which determines the phenotype when present in the heterozygous or homozygous state. The intensity of the phenotype is however generally higher when the allele is present in the homozygous state.

A “recessive” allele refers to an allele which determines the phenotype when present in the homozygous state only.

“Backcrossing” is understood within the scope of the invention to refer to a process in which a hybrid progeny is repeatedly crossed back to one of the parents. Different recurrent parents may be used in subsequent backcrosses.

“Locus” is understood within the scope of the invention to refer to a region on a chromosome, which comprises a gene, a QTL or its corresponding genetic sequence contributing to a trait.

“Genetic linkage” is understood within the scope of the invention to refer to an association of characters in inheritance due to location of genes in proximity on the same chromosome, measured by percent recombination between loci (centi-Morgan, cM).

As used herein, the phrases “sexually crossed” and “sexual reproduction” in the context of the presently disclosed subject matter refers to the fusion of gametes to produce progeny (e.g., by fertilization, such as to produce seed by pollination in plants). A “sexual cross” or “cross-fertilization” refers to, in some embodiments, fertilization of one individual by another (e.g., cross-pollination in plants). The term “selfing” refers, in some embodiments, to the production of seed by self-fertilization or self-pollination, i.e., pollen and ovule are from the same plant.

As used herein, the phrase “genetic marker” or “DNA marker” refers to a feature of an individual's genome (e.g., a nucleotide or a polynucleotide sequence that is present in an individual's genome) that is associated with one or more loci of interest. In some embodiments, a genetic marker is polymorphic in a population of interest, or the locus occupied by the polymorphism, depending on context. Genetic markers include, for example, single nucleotide polymorphisms (SNPs), indels (i.e., insertions/deletions), simple sequence repeats (SSRs), restriction fragment length polymorphisms (RFLPs), random amplified polymorphic DNAs (RAPDs), cleaved amplified polymorphic sequence (CAPS) markers, Diversity Arrays Technology (DArT) markers, and amplified fragment length polymorphisms (AFLPs), among many other examples. Genetic markers can, for example, be used to locate genetic loci containing alleles on a chromosome that contribute to variability of phenotypic traits. The phrase “genetic marker” can also refer to a polynucleotide sequence complementary to a genomic sequence, such as a sequence of a nucleic acid used as probes.

As used herein, the term “genotype” refers to the genetic constitution of a cell or organism. An individual's “genotype for a set of genetic markers” includes the specific alleles, for one or more genetic marker loci, present in the individual's haplotype.

As used herein, the term “progeny” refers to the descendant(s) of a particular cross. Typically, progeny result from breeding of two individuals, although some species (particularly some plants and hermaphroditic animals) can be selfed (i.e., the same plant acts as the donor of both male and female gametes). The descendant(s) can be, for example, of the F1, the F2, or any subsequent generation.

As used herein, the terms “quantitative trait locus” (QTL) refer to an association between a genetic marker and a chromosomal region and/or gene and/or introgressed sequence that affects the phenotype of a trait of interest. Typically, this is determined statistically, e.g., based on one or more methods published in the literature. A QTL can be a chromosomal region and/or a genetic locus with at least two alleles that differentially affect a phenotypic trait.

The term “recipient tomato plant” is used herein to indicate a tomato plant that is to receive DNA obtained from a donor tomato plant that comprises the improved ToBRFV resistance trait.

The term “natural genetic background” is used herein to indicate the original genetic background of genetic sequence. For instance, the genetic sequence of the present invention was found at a specific location on chromosome 1 of a Solanum galapagense plant. Conversely, a method that involves the transfer of DNA, via e.g., breeding, comprising this genetic sequence from chromosome 1 of Solanum galapagense plant to the same position on chromosome 1 of another tomato species, preferably a cultivated tomato plant, even more preferably a Solanum lycopersicum plant, will result in this genetic sequence not being in its natural genetic background. When the genetic sequence of the present invention is transferred from a Solanum galapagense background into another tomato species, preferably a cultivated tomato plant, even more preferably a Solanum lycopersicum plant, they are referred to as “introgressed sequence” or “introgressed genetic sequence”.

A “donor tomato plant” is understood within the scope of the invention to mean the tomato plant which provides the improved ToBRFV resistance trait.

“Marker-based selection” is understood within the scope of the invention to refer to e.g. the use of genetic markers to detect one or more nucleic acids from the plant, where the nucleic acid is associated with a desired trait to identify plants that carry alleles for desirable (or undesirable) traits, so that those plants can be used (or avoided) in a selective breeding program.

A single nucleotide polymorphism (SNP), a variation at a single site in DNA, is the most frequent type of variation in the genome. A single-nucleotide polymorphism (SNP) is a DNA sequence variation occurring when a single nucleotide—A, T, C, or G—in the genome (or other shared sequence) differs between members of a biological species or paired chromosomes in an individual. For example, two sequenced DNA fragments from different individuals, AAGCCTA to AAGCTTA, contain a difference in a single nucleotide. In this case there are two alleles: C and T. The basic principles of SNP array are the same as the DNA microarray. These are the convergence of DNA hybridization, fluorescence microscopy, and DNA capture. The three components of the SNP arrays are the array that contains nucleic acid sequences (i.e., amplified sequence or target), one or more labelled allele-specific oligonucleotide probes and a detection system that records and interprets the hybridization signal. The presence or absence of the desired SNP marker allele may be determined by real-time PCR using double-stranded DNA dyes or the fluorescent reporter probe method.

“PCR (Polymerase chain reaction)” is understood within the scope of the invention to refer to a method of producing relatively large amounts of specific regions of DNA or subset(s) of the genome, thereby making possible various analyses that are based on those regions. “PCR primer” is understood within the scope of the invention to refer to relatively short fragments of single-stranded DNA used in the PCR amplification of specific regions of DNA.

“Probe” as used herein refers to a group of atoms or molecules which can recognise and bind to a specific target molecule or cellular structure and thus allowing detection of the target molecule or structure. Particularly, “probe” refers to a labelled DNA or RNA sequence which can be used to detect the presence of and to quantitate a complementary sequence by molecular hybridization.

“Sequence Identity”. The terms “identical” or “identity” in the context of two or more nucleic acid or protein sequences, refer to two or more sequences or sub-sequences that are the same or have a specified percentage of amino acid residues or nucleotides that are the same, when compared and aligned for maximum correspondence, as measured using one of the following sequence comparison algorithms or by visual inspection. If two sequences which are to be compared with each other differ in length, sequence identity preferably relates to the percentage of the nucleotide residues of the shorter sequence which are identical with the nucleotide residues of the longer sequence. As used herein, the percent identity/homology between two sequences is a function of the number of identical positions shared by the sequences (i.e., % identity=# of identical positions/total #of positions×100), considering the number of gaps, and the length of each gap, which need to be introduced for optimal alignment of the two sequences. The comparison of sequences and determination of percent identity between two sequences can be accomplished using a mathematical algorithm, as described herein below. For example, sequence identity can be determined conventionally with the use of computer programs such as the Bestfit program (Wisconsin Sequence Analysis Package, Version 8 for Unix, Genetics Computer Group, University Research Park, 575 Science Drive Madison, WI 53711). Bestfit utilizes the local homology algorithm of Smith and Waterman, Advances in Applied Mathematics 2 (1981), 482-489, to find the segment having the highest sequence identity between two sequences. When using Bestfit or another sequence alignment program to determine whether a particular sequence has for instance 95% identity with a reference sequence of the present invention, the parameters are preferably so adjusted that the percentage of identity is calculated over the entire length of the reference sequence and that homology gaps of up to 5% of the total number of the nucleotides in the reference sequence are permitted. When using Bestfit, the so-called optional parameters are preferably left at their pre-set (“default”) values. The deviations appearing in the comparison between a given sequence and the above-described sequence of the invention may be caused for instance by addition, deletion, substitution, insertion or recombination. Such a sequence comparison can preferably also be carried out with the program “fasta20u66” (version 2.0u66, September 1998 by William R. Pearson and the University of Virginia; see also W.R. Pearson (1990), Methods in Enzymology 183, 63-98, appended examples and http://workbench.sdsc.edu/). For this purpose, the “default” parameter settings may be used.

### Embodiments

### Plants, Seeds, Fruits.

In a first embodiment, the invention provides a cultivated tomato plant, preferably a cultivated Solanum lycopersicum plant resistant to ToBRFV infection, comprising in its genome an introgressed sequence from Solanum galapagense which confers resistance to ToBRFV, wherein said introgressed sequence is located on chromosome 1 and comprises at least one of the following SNP markers:
a) an A genotype in the homozygous state for SNP marker 2 at a position corresponding to position 79 in SEQ ID NO: 6;
b) a G genotype in the homozygous state for SNP marker 3 at a position corresponding to position 51 in SEQ ID NO: 11; and/or
c) a G genotype in the homozygous state for SNP marker 4 at a position corresponding to position 147 in SEQ ID NO: 16.

Further, the plant of the previous embodiment wherein:
a) the A genotype for SNP marker 2 can be identified in a PCR by amplification of a nucleic acid fragment with a pair of oligonucleotide primers: forward primer of SEQ ID NO: 7 and reverse primer of SEQ ID NO: 10, and probe of SEQ ID NO: 8;
b) the G genotype for SNP marker 3 can be identified in a PCR by amplification of a nucleic acid fragment with a pair of oligonucleotide primers: forward primer of SEQ ID NO: 12 and reverse primer of SEQ ID NO: 15, and probe of SEQ ID NO: 13; and/or
c) the G genotype for SNP marker 4 can be identified in a PCR by amplification of a nucleic acid fragment with a pair of oligonucleotide primers: forward primer of SEQ ID NO: 17 and reverse primer of SEQ ID NO: 20, and probe of SEQ ID NO: 18.

In a further embodiment of the invention, said ToBRFV resistance-conferring introgressed sequence comprises at least one of SEQ ID NO: 6, SEQ ID NO: 11, and/or SEQ ID NO: 16, or a sequence that is at least 80%, preferably at least 85%, more preferably at least 90%, even more preferably at least 95% identical to one or more of said sequences.

In a further embodiment of the invention, said plant comprises SEQ ID NO: 11.

In a further embodiment of the invention, said introgressed sequence is comprised in tomato plant 19TEP080670, representative seed of which is deposited under NCIMB Accession No. 43938, or a progeny or an ancestor thereof.

In a further embodiment, the invention provides a plant according to any of the preceding embodiments wherein said plant is obtained by crossing tomato plant 19TEP080670, representative seed of which is deposited under NCIMB Accession No. 43938, or a progeny or an ancestor thereof, with a tomato plant that does not contain said ToBRFV resistance-conferring introgressed sequence.

In a further embodiment, the invention provides a plant according to any of the preceding embodiments, wherein said plant is an inbred, a dihaploid, a diploid, or a hybrid plant.

In another embodiment, the plant according to the invention is male sterile. In another embodiment, the plant according to the invention is cytoplasmic male sterile.

In a further embodiment, the tomato plant of the invention is a tomato plant according to any of preceding embodiments, wherein said ToBRFV resistance-conferring introgressed sequence located on chromosome 1 can be identified using any of the SNP markers 2 to 4 disclosed in Table 4 hereinbelow.

In a further embodiment, the invention provides a cultivated tomato plant, preferably a cultivated Solanum lycopersicum plant resistant to ToBRFV infection, comprising in its genome an introgressed sequence from Solanum galapagense which confers resistance to ToBRFV located on chromosome 1, wherein said plant genome comprises:
a) an A genotype in the homozygous state for SNP marker 1 at a position corresponding to position 47 in SEQ ID NO: 1, and
b) a C genotype in the heterozygous or homozygous state for SNP marker 5 at a position corresponding to position 94 in SEQ ID NO: 21; and
c) at least one of the resistant genotypes at any one of the SNP markers 2 to 4 disclosed in Table 4.

In a further embodiment, the cultivated tomato plant of the previous embodiment further comprises at least a second resistant genotype at any of the SNP markers 2 to 4 disclosed in Table 4.

In a further embodiment, the invention provides a cultivated tomato plant, preferably a cultivated Solanum lycopersicum plant resistant to ToBRFV infection, comprising in its genome an introgressed sequence from Solanum galapagense which confers resistance to ToBRFV located on chromosome 1, wherein said plant genome comprises:
a) a G genotype in the homozygous state for SNP marker 3 at a position corresponding to position 51 in SEQ ID NO: 11.

In a further embodiment, the tomato plant of the invention is a tomato plant according to any of the preceding embodiments, wherein tomato plant 19TEP080670, representative seed of which is deposited under NCIMB Accession No. 43938, or a progeny or an ancestor thereof, or Solanum galapagense accession LA0483, is the source of said ToBRFV resistance-conferring introgressed sequence.

It is a further embodiment to provide a plant part, organ or tissue obtainable from a tomato plant according to any of preceding embodiments, including but not limiting to leaves, stems, roots, flowers or flower parts, fruits, shoots, gametophytes, sporophytes, pollen, anthers, microspores, egg cells, zygotes, embryos, meristematic regions, callus tissue, seeds, cuttings, cell or tissue cultures or any other part or product of the plant which still exhibits the ToBRFV resistance trait according to the invention, particularly when grown into a plant that produces fruits.

In a further embodiment, the invention provides a seed that produces a plant according to any of the preceding embodiments.

In a further embodiment the invention relates to the use of a tomato plant according to any of the preceding embodiments as a rootstock, preferably a tomato rootstock. In a further embodiment the invention relates to the use of tomato plant 19TEP080670, representative seed of which is deposited under NCIMB Accession No. 43938, or a progeny or an ancestor thereof, as a tomato rootstock.

In another embodiment is considered the use of a tomato plant, plant part or seed according to any of the preceding embodiments for producing and harvesting tomato fruits.

In another embodiment the invention relates to the use of a tomato plant, plant part or seed according to any embodiments, wherein the tomato plant, plant part or seed is tomato plant 19TEP080670, representative seed of which is deposited under NCIMB Accession No. 43938, or a progeny or an ancestor thereof.

In a further embodiment the invention relates to the use of a tomato plant, plant part or seed according to any of the preceding embodiments to sow a field, a greenhouse, or a plastic house.

In one embodiment, the invention provides tomato fruits produced by a tomato plant according to any of the preceding embodiments.

The invention further relates to the use of a tomato plant according to any of the preceding embodiments to introgress a ToBRFV resistance trait into a tomato plant lacking said ToBRFV resistance trait.

In a further embodiment, the tomato plant of any preceding embodiment is resistant to Tobamoviruses. In a further embodiment, said tomato plant is also resistant to TMV and ToMV viruses. In a further embodiment, said tomato plant is also resistant to ToMV pathotypes 0 and 2.

### Genetic Sequences, Markers.

The present invention is further directed to an introgressed genetic sequence linked to the ToBRFV resistance trait in the tomato plant. In a further embodiment, the genetic sequence of the present invention is located on chromosome 1. In a further embodiment of the present invention, the genetic sequence is comprised in, obtained from or obtainable from a donor plant of tomato plant 19TEP080670, representative seed of which is deposited under NCIMB Accession No. 43938, or a progeny or an ancestor thereof, and comprising said genetic sequence, or from Solanum galapagense accession LA0483.

In another embodiment, the introgressed genetic sequence of the present invention is located on chromosome 1 and is characterized by at least one of the following resistance genotypes at one of the SNP markers selected in the list comprising:
a) an A genotype in the homozygous state for SNP marker 2 at a position corresponding to position 79 in SEQ ID NO: 6;
b) a G genotype in the homozygous state for SNP marker 3 at a position corresponding to position 51 in SEQ ID NO: 11; and/or
c) a G genotype in the homozygous state for SNP marker 4 at a position corresponding to position 147 in SEQ ID NO: 16.

The present invention also discloses the use of at least one, at least two or at least three of the SNP markers according to the invention for diagnostic selection and/or genotyping of the ToBRFV resistance trait locus in a tomato plant, particularly a cultivated tomato plant.

The present invention further discloses the use of at least one, at least two or at least three of the SNP markers according to the invention for identifying in a tomato plant, particularly a cultivated tomato plant, more particularly a tomato plant according to the invention, the presence of the ToBRFV resistance trait and/or for monitoring the introgression of the ToBRFV resistance trait in a tomato plant, particularly a cultivated tomato plant, particularly a tomato plant according to the invention and as described herein.

It is further disclosed that the introgressed sequence of the present invention provides broad spectrum resistant against Tobamoviruses, preferably against TMV and ToMV viruses, more preferably against ToMV pathotypes 0 and 2.

The present invention therefore further relates in one embodiment to derived markers, particularly to derived primers or probes, developed from an amplification product according to the invention and as described herein above by methods known in the art, which derived markers are genetically linked to the ToBRFV resistance trait locus.

### Methods of Breeding.

In a further embodiment, the invention provides a method for producing a cultivated tomato plant, preferably a cultivated Solanum lycopersicum plant resistant to ToBRFV infection comprising the steps of
a) crossing a plant according to any one of the preceding embodiments with a cultivated tomato plant lacking said ToBRFV resistance-conferring introgressed sequence;
b) selecting a progeny plant comprising said introgressed sequence located on chromosome 1 conferring resistance to ToBRFV, said selecting step comprising detecting a resistance genotype for at least one of the following SNP markers:
   i) an A genotype in the homozygous state for SNP marker 2 at a position corresponding to position 79 in SEQ ID NO: 6;
   ii) a G genotype in the homozygous state for SNP marker 3 at a position corresponding to position 51 in SEQ ID NO: 11; and/or
   iii) a G genotype in the homozygous state for SNP marker 4 at a position corresponding to position 147 in SEQ ID NO: 16;
      thereby producing a plant with enhanced resistance to ToBRFV.

In a further embodiment, the invention relates to the method of any of the preceding embodiments, wherein the method further comprises:
c) selfing the selected progeny or crossing the selected progeny with another tomato plant to produce further progeny.

In a further embodiment, the invention relates to the method of the preceding embodiment, wherein further progeny is selected and selfed/crossed for 2 to 10 more generations.

In a further embodiment, the invention relates to the method of any of the preceding embodiments, wherein the plant of step a) is tomato plant 19TEP080670, representative seed of which is deposited under NCIMB Accession No. 43938, or a progeny or an ancestor thereof, or Solanum galapagense accession LA0483.

In another embodiment the invention relates to a method of providing a ToBRFV resistant tomato plant, plant part or seed, wherein said method comprises the following steps:
a) Crossing a 1^{st} plant lacking the ToBRFV resistance-conferring introgressed sequence of the invention with a 2^{nd} tomato plant according to any embodiments,
b) Obtaining a progeny tomato plant, and,
c) Optionally, selecting a plant of said progeny characterized in that said plant exhibits resistance to ToBRFV.

In a further embodiment the invention relates to the method of the preceding embodiment wherein the 2^{nd} tomato plant is tomato plant 19TEP080670, representative seed of which is deposited under NCIMB Accession No. 43938, or a progeny or an ancestor thereof or Solanum galapagense accession LA0483.

In another embodiment the invention relates to a method for producing a ToBRFV resistant tomato plant comprising the following steps:
a) Providing seeds of a tomato plant according to any of the preceding embodiments,
b) Germinating said seed and growing a mature, fertile plant therefrom,
c) Inducing self-pollination of said plant under a), growing fruits and harvesting the fertile seeds therefrom, and
d) Growing plants from the seeds harvested under c) and selecting a ToBRFV resistant tomato plant.

In another embodiment the invention relates to a method for increasing the resistance to ToBRFV of a tomato plant, comprising the steps of:
a) selecting a tomato, which comprises a ToBRFV resistance trait associated with one introgressed sequence located on chromosome 1, wherein said trait can be identified by the presence of a resistance genotype at least one of the SNP markers listed in Table 4;
b) crossing said plant of step a), which comprises a ToBRFV resistance trait, with a tomato plant, particularly a cultivated tomato plant, which does not comprise a ToBRFV resistance trait and shows susceptibility to ToBRFV, as compared to the plant of step a), and
c) selecting progeny from said cross which shows increased ToBRFV resistance, as compared to the plant of step b).

In a further embodiment, the invention relates to a method for producing a F1 tomato plant exhibiting resistance to ToBRFV, the method comprising crossing an inbred tomato plant, which is a plant according to any one of the preceding embodiments, with a different inbred tomato plant to produce F1 hybrid progeny.

### Methods of Selection.

In a further embodiment, the invention provides a method for identifying a cultivated tomato plant, preferably a cultivated Solanum lycopersicum plant, exhibiting resistance to ToBRFV and having at least one copy of said ToBRFV resistance-conferring introgressed sequence, said method comprising the step of detecting at least one resistance genotype for at least one of the following SNP markers:
a) an A genotype in the homozygous state for SNP marker 2 at a position corresponding to position 79 in SEQ ID NO: 6;
b) a G genotype in the homozygous state for SNP marker 3 at a position corresponding to position 51 in SEQ ID NO: 11; and/or
c) a G genotype in the homozygous state for SNP marker 4 at a position corresponding to position 147 in SEQ ID NO: 16;
   thereby identifying a tomato plant exhibiting resistance to ToBRFV.

In a further embodiment, the invention relates to the method of the preceding embodiment, wherein said method further comprises selecting a tomato plant comprising said one or more resistance genotypes at the corresponding SNP markers, and crossing the selected tomato plant with a second tomato plant to produce progeny tomato plants that comprise at least one resistance genotype for at least one of said SNP markers and exhibits resistance to ToBRFV.

In another embodiment the invention relates to a method of identifying a tomato plant comprising the ToBRFV resistance-conferring introgressed sequence of the invention, wherein said method comprises the steps of:
a) providing a population segregating for the ToBRFV resistance trait,
b) screening the segregating population for a member exhibiting resistance to ToBRFV, wherein said trait can be identified by the presence of ToBRFV resistance-conferring introgressed sequence of the invention,
c) selecting one member of the segregating population, wherein said member comprises the ToBRFV resistance trait.

In a further embodiment, the invention provides a method for identifying a cultivated tomato plant comprising an introgressed sequence on chromosome 1, wherein said introgressed sequence confers resistance to ToBRFV, comprising:
a) providing a population segregating for ToBRFV resistance,
b) screening said population using a kit which detects at least one resistance genotype for at least one of the SNP markers listed in Table 4, and,
c) identifying a plant comprising at least one resistance genotype for at least one SNP marker selected in the list of Table 4.

In a further embodiment, the invention provides a method for identifying a tomato source of ToBRFV resistance trait on chromosome 1, comprising:
a) providing a tomato accession or a plurality of tomato accessions,
b) screening said tomato accession or plurality of tomato accessions using a kit which detects at least one resistance genotype for at least one of the SNP markers listed in Table 4, and,
c) identifying a wild tomato accession comprising said one resistance genotype for at least one SNP marker selected in the list of Table 4.

In yet another embodiment, the invention relates to the use of at least one SNP marker amplified from the genome of a tomato plant according to any of the preceding embodiments, preferably from the genome of tomato plant 19TEP080670, representative seed of which is deposited under NCIMB Accession No. 43938, or a progeny or an ancestor thereof, or Solanum galapagense accession LA0483, wherein said SNP marker is identified using one of the SNP markers listed in Table 4 and wherein said SNP marker is indicative of the presence of the ToBRFV resistance trait in a tomato plant, to identify a tomato plant that comprises and exhibits the ToBRFV resistance trait.

In a further embodiment, the invention relates to a method for assessing the genotype of a cultivated tomato plant, preferably a Solanum lycopersicum plant, exhibiting resistance to ToBRFV, said method comprising the steps of:
a) providing a sample from said plant, and,
b) detecting in said sample a QTL locus located on chromosome 1 and associated with said ToBRFV resistance, said QTL locus being flanked by SNP markers 1 and 5, and at least one of the following SNP markers:
   i) an A genotype in the homozygous state for SNP marker 2 at a position corresponding to position 79 in SEQ ID NO: 6;
   ii) a G genotype in the homozygous state for SNP marker 3 at a position corresponding to position 51 in SEQ ID NO: 11;
   iii) a G genotype in the homozygous state for SNP marker 4 at a position corresponding to position 147 in SEQ ID NO: 16; and/or
   iv) any other DNA marker associated with said QTL locus flanked by SNP markers 1 and 5.

In a further embodiment, the invention relates to a method of identifying in a cultivated tomato plant, preferably a cultivated Solanum lycopersicum plant, an introgressed sequence associated with an increased resistance to ToBRFV, said method comprising the step of detecting in said plant an allele of at least one DNA marker that is genetically linked to a QTL locus associated with said increased resistance to ToBRFV, wherein said allele maps within 10 cM, preferably within 5 cM of said QTL locus located on chromosome 1 in a genomic region flanked by SNP markers 1 and 5.

In a further embodiment, the invention relates to the method of the preceding embodiment, wherein said QTL locus can be identified by at least one of the following SNP markers
a) an A genotype in the homozygous state for SNP marker 2 at a position corresponding to position 79 in SEQ ID NO: 6;
b) a G genotype in the homozygous state for SNP marker 3 at a position corresponding to position 51 in SEQ ID NO: 11; and/or
c) a G genotype in the homozygous state for SNP marker 4 at a position corresponding to position 147 in SEQ ID NO: 16.

In a further embodiment, the invention relates to the method of the preceding embodiment, wherein said method further comprises the step of selecting a cultivated tomato plant, preferably a cultivated Solanum lycopersicum plant comprising said introgressed sequence.

In a further embodiment, the invention relates to a method of identifying a cultivated tomato plant, preferably a cultivated Solanum lycopersicum plant, exhibiting increased resistance to ToBRFV by identifying a QTL associated with said increased resistance to ToBRFV, the method comprising the steps of:
a) detecting at least one DNA marker from a tomato plant, which DNA marker is linked to a chromosomal interval associated with increased resistance to ToBRFV, wherein said chromosomal interval is flanked on each side by SNP markers having at least 80% sequence identity to SEQ ID NOs: 1 and 21; and
b) identifying said tomato plant comprising said at least one DNA marker.

### Uses.

The present invention also relates to the use of ToBRFV resistance-propagating material obtainable from a tomato plant according to any of the preceding embodiments for growing a tomato plant to produce ToBRFV resistant tomato plants wherein said ToBRFV resistance may be assessed in a standard assay, particularly an assay as described in Example 2 below.

The present invention also relates to the use of ToBRFV resistance propagating material obtainable from a tomato plant according to any of the preceding embodiments for producing tomato fruits.

In another embodiment the invention relates to the use a cultivated tomato plant, plant part or seed, more preferably a cultivated Solanum lycopersicum plant, plant part or seed according to any of the preceding embodiments for growing a plant and producing and harvesting crops and/or fruits.

In another embodiment the invention relates to the use of a cultivated tomato plant, more preferably a cultivated Solanum lycopersicum plant, according to any of the preceding embodiments for producing fruits for the fresh market or for food processing.

In another embodiment the invention relates to the use of a cultivated tomato plant, plant part or seed, preferably a cultivated Solanum lycopersicum plant, plant part or seed according to any of preceding embodiments, wherein said cultivated tomato plant, plant part or seed, preferably the cultivated Solanum lycopersicum plant, plant part or seed is of tomato plant 19TEP080670, representative seed of which is deposited under NCIMB Accession No. 43938, or a progeny or an ancestor thereof.

In a further embodiment the invention relates to the use of a cultivated tomato plant, plant part or seed, more preferably a cultivated Solanum lycopersicum plant, plant part or seed according to any of the preceding embodiments to sow a field, a greenhouse, or a plastic house.

In a further embodiment the invention relates to the use of a tomato plant according to any of the preceding embodiments to confer the increased ToBRFV resistance trait to a tomato plant lacking said trait. The invention further relates to the use of a tomato plant according to any of the preceding embodiments to introgress an increased ToBRFV resistance trait into a tomato plant lacking said trait. The invention further relates to the use of a tomato plant according to any of the preceding embodiments to introgress an increased Tobamoviruses resistance trait into a tomato plant lacking said trait. The invention further relates to the use of a tomato plant according to any of the preceding embodiments to introgress an increased ToMV pathotypes 0 and 2 resistance trait into a tomato plant lacking said trait.

In a further embodiment the invention relates to the use of any of SEQ ID NOs 1-25 for screening a population of tomato plants for the presence of a QTL locus located on chromosome 1 and associated with an increased ToBRFV resistance.

In a further embodiment the invention relates to the use of SEQ ID NO 6, 11 and/or 16 for screening a population of tomato plants for the presence of a QTL locus located on chromosome 1 and associated with an increased ToBRFV resistance.

Based on the description of the present invention, the skilled person who is in possession of tomato plant 19TEP080670, representative seed of which is deposited under NCIMB Accession No. 43938, or a progeny thereof, comprising said introgressed genetic sequence, as described herein, has no difficulty to transfer the said introgressed genetic sequence of the present invention to other tomato plants of various types using breeding techniques well-known in the art with the support of SNP markers herein disclosed.

### Seed Deposit Details

Applicant has made a deposit of 625 seeds of tomato plant 19TEP080670 with NCIMB (National Collection of Industrial, Food and Marine Bacteria, NCIMB Limited, Ferguson Building, Craibstone Estate, Bucksburn, Aberdeen AB21 9YA, Scotland) on 11 Feb. 2022 under NCIMB Accession No. 43938.

Applicant elects for the expert solution and requests that the deposited material be released only to an Expert according to Rule 32(1) EPC or corresponding laws and rules of other countries or treaties (Expert Witness clause), until the mention of the grant of the patent publishes, or from 20 years from the date of filing if the application is refused, withdrawn, or deemed to be withdrawn.

Tomato hybrid plant 19TEP080670 is heterozygous for the ToBRFV resistance QTL on chromosome 1, i.e., tomato plant 19TEP080670 comprises one copy of the introgressed sequence on chromosome 1 that is associated with increased ToBRFV resistance.

### EXAMPLES

### Example 1

### Germplasm And Population Development

Solanum galapagense accession LA0483 was identified in a ToBRFV screen as exhibiting increased ToBRFV resistance and was therefore used as a source for the creation of a discovery population.

An F1 population resulting from a cross between LA0483 and a susceptible elite line 16TEP070330 was self-pollinated to get 20 F2 lines.

One of these F2 lines, 20TEP070778, was used to perform a QTL mapping based on genotyping and phenotyping data of individual F2 plants. A total of 443 F2 plants were sampled and genotyped to establish a genetic map of the population. In parallel, a subset of 349 plants were mechanically inoculated with a ToBRFV strain on seedlings and assessed according to a qualitative phenotyping score described in Example 2A-C below. A QTL associated with increased ToBRFV resistance located on chromosome 1 was identified in this F2 population (see Examples 3 to 5).

One progeny of the initial tomato cross, 19TEP080670, was retained and deposited at NCIMB on 11 Feb. 2022 under NCIMB Accession No. 43938. Tomato plant 19TEP080670 is heterozygous for the ToBRFV resistance trait, i.e., it comprises one copy of the ToBRFV resistance-conferring introgressed sequence from LA0483.

### Example 2

### Protocols

### Example 2A

### ToBRFV Isolate

A ToBRFV strain was collected in Israel in 2015 and thereafter stored on dried tomato leaves. From 1 gram of these tomato leaves, an inoculum production was performed by crushing the leaves and adding 5 ml of buffer, 0.1 gram of charcoal and 0.1 gr of carborundum (abrasive dust). The obtained solution was used to mechanically inoculate cotyledons of susceptible materials. Trays containing seedlings were maintained in growth chamber at 18° C. during night/24° C. during day for 10-14 days to increase inoculum. Day length was fixed at for 15 hours under full light (around 10 000 lux). Symptomatic leaves were then harvested from the seedlings and stored in a −80° C. freezer. This storage is considered as the source of inoculum. Following this production, a first step of calibration was performed to establish the lower concentration of inoculum allowing to infect 100% of susceptible controls, without escape. The best dilution fitting with this requirement was fixed at ½ (1 g viral leaves in 10 ml buffer) and was used in the phenotyping experiment.

### Example 2B

### Preparation and Inoculation of Plants

An artificial method was used to inoculate plants with the ToBRFV strain. Seeds of the F2 lines were sown in specific trays and placed in a growth room in Sarrians, France. In addition, twenty-four seeds of parental lines 16TEP070330 and LA0483, resulting F1 plant 19TEP080670 and susceptible control 16TEP071665 were sown to use as checks. Four days before inoculation, depending on germination rate, 349 plants of F2 and 16 plants of checks were selected for inoculation. For each material tested, 1 plant was transplanted in another support. Plants in trays were inoculated 14 days (2 true leaves) after sowing using ToBRFV inoculum prepared as described above. The inoculation was done on by rubbing gently twice the leaves. Transplanting plants were inoculated without virus (mock) to evaluate the inoculation effect.

### Example 2C

### Scoring of ToBRFV Resistance

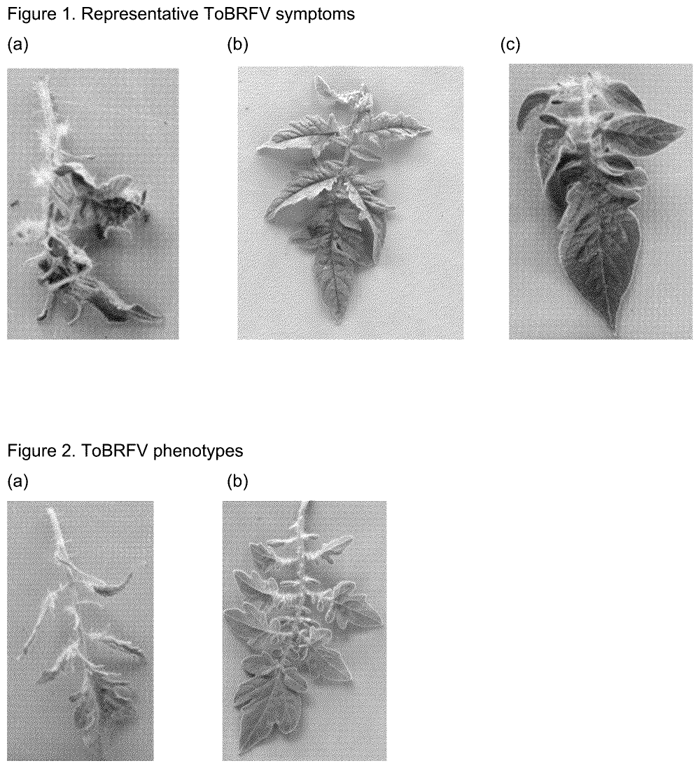

The first symptoms such as distortion and mosaic on the leaves appeared 16-19 days post-inoculation (dpi). Plants were monitored and symptoms were assessed when susceptible checks are fully symptomatic, in this case at 20 and 27 dpi. Plants were scored in a qualitative scale as described below and illustrated in FIG. 1.
Rating Symptoms
HR (9) Healthy plants with no symptoms.
IR (5) Low symptoms expression compared to the susceptible
S (1) Susceptible plants with strong leaves distortion.

### Example 2D

### Method of Identifying the QTL and Corresponding Introgressed Sequence Underlying the ToBRFV Increased Resistance Trait

For QTL discovery, 349 plants of the “LA0483×16TEP070330” population were genotyped with 191 genetic markers spanning the genome. These plants were grown and evaluated for ToBRFV as described in Example 2A-C above.

The QTL detection was performed using the R/qtl package in the R statistical framework. First, the function ‘calc.genoprob’ was used to calculate the genotype probabilities (step 1 cM). Haley-Knott regression was performed to provide an approximation of the results of standard interval mapping. Then, the function ‘stepwiseqtl’ was invoked, which provides a fully automated model selection forward/backward algorithm. LOD threshold for main effect was determine by 10,000 permutations. The function ‘fitqtl’ was used to fit the final QTL model and the function qtlStats' to obtain estimates of QTL effects.

### Example 3

### Identification of One QTL Associated with Increased TOBREV Resistance

One QTL was identified based on the ToBRFV resistance phenotypes from the F2 population. Table 1 shows the chromosomal location, the effect of the QTL measured as LOD score, and the percentage of variation explained by the QTL on chromosome 1 for ToBRFV resistance. The QTL showed a recessive effect in the F2 population.

TABLE 1
Significant QTL associated with ToBRFV resistance.

| Chromosome | LOD | % var | Pvalue (F) |
|---|---|---|---|
| 1 | 9.98 | 9.64% | 4.24E−08 *** |

“LOD” = log likelihood score, “% var” = percent phenotypic variation explained by the QTL, “Pvalue (F)” = the probability of the QTL detected due to random chance by F test.

### Example 4

### Assessment of the TOBRFV Resistance in a Panel of Tomato Plants

Solanum galapagense accession LA0483 is a wild tomato plant with a smelly foliage, a bushier habit and small, orange, hairy fruits. The susceptible material 16TEP070330 is an elite line from an indeterminate cluster tomato type for passive greenhouse cropping.

The LA0483 source presented a strong resistance to ToBRFV, whereas 16TEP070330 susceptible parent and the F1 resulting from the ‘LA0483×16TEP070330’ cross exhibit severely susceptible phenotypes. F2 plants carrying the genetic sequence associated with increased resistance to ToBRFV present on chromosome 1 of Solanum galapagense accession LA0483 in homozygous state presented a strong resistant phenotype. On the other hand, F2 plants heterozygous in this same genomic region or carrying two copies of the susceptible 16TEP070330 alleles exhibit susceptible phenotypes.

The phenotyping results, along with the results of testing for the presence or absence of representative markers in QTL1, are summarized in Table 2 below.

TABLE 2
Presence or absence of flanking and characterizing SNP
markers for QLT1 and corresponding ToBRFV phenotypes.

| | | ToBRFV | QLT1 region | | | | |
|---|---|---|---|---|---|---|---|
| Material No, ID and type | | score | ST2752 | ST4450 | ST1840 | ST4079 | ST0435A |
| 1. | LA0483: Original donor | HR | G/G | A/A | G/G | G/G | A/A |
| 2. | 16TEP070330: Susceptible | S | A/A | G/G | A/A | A/A | C/C |
| | parent | | | | | | |
| 3. | F1, ‘LA0483 × 16TEP070330’, | S | G/A | A/G | G/A | G/A | A/C |
| | 19TEP080670, seed deposit | | | | | | |
| 4. | F2 line, DR225602385 | S | A/A | G/G | A/A | A/A | C/C |
| 5. | F2 line, DR225602252 | S | A/A | G/G | A/A | A/A | C/C |
| 6. | F2 line, DR225602575 | S | G/A | A/G | G/A | G/A | A/C |
| 7. | F2 line, DR225602168 | S | G/A | A/G | G/A | G/A | A/C |
| 8. | F2 line, DR225602183 | HR | G/G | A/A | G/G | G/G | A/A |
| 9. | F2 line, DR225602191 | HR | G/G | A/A | G/G | G/G | A/A |

Existing susceptible check plant (No 2) as well as the F1 plant (No 3) and the F2 lines comprising 0 (No 4 and 5) or 1 copy of the introgressed sequence at QTL1 (No 6 and 7) exhibited susceptible phenotypes. On the other end, the original donor plant (No 1) and the F2 lines comprising 2 copies of the introgressed sequence at QTL1 (No 8 and 9) bearing the resistance genotypes at the SNP markers spanning said introgressed sequence exhibited an increased ToBRFV resistance, achieving a High Resistance score.

Within the QTL1 region, three SNP markers, ST4450, ST1840 and ST4079 (SNP markers 2,3 and 4 respectively) within the QTL interval showed specificity for the selection of donor resistant allele from the resistance donor, and from them, SNP marker ST1840 (SNP marker 3) is the most closely linked to the resistance.

Table 3 shows both genetic and physical positions of the QTL on chromosome 1 as well as the positions of the three SNP markers tightly linked with the QTL. Physical positions are provided with reference to the public genome assembly of Heinz 1706 SL4.0 (released September 2019, https://solgenomics.net/).

TABLE 3
Genetic map of the QTL on chromosome 1

| | | | Physical position | |
|---|---|---|---|---|
| SNP | SNP | Position | (bp; Heinz | |
| ID | Locus | (cM) | 1706 SL4.0) | Observation |
| 1 | ST2752 | 86.18 | 82,311,506 | Left flanking marker |
| 2 | ST4450 | 93.4 | 84,759,575 | SNP specific to R allele |
| 3 | ST1840 | 97.17 | 86,137,823 | SNP specific to R allele |
| 4 | ST4079 | 103.74 | 87,456,377 | SNP specific to R allele |
| 5 | ST0435A | 110.57 | 88,906,129 | Right flanking marker |

### Example 5

### Sequence and SNP Marker Information For QLT1

The sequence information of SNP markers 1 to 5 (ST2752, ST4450, ST1840, ST4079 and ST0435A) is summarized in Table 4 below.

TABLE 4

| MARKER | 1/ ST2752 | 2/ ST4450 | 3/ ST1840 | 4/ ST4079 | 5/ ST0435A |
|---|---|---|---|---|---|
| Resistant Allele (S. galapagense) | G | A | G | G | A |
| Susceptible Allele (S. lycopersicum) | A | G | A | A | C |
| Target Sequence: SEQ ID NO. | 1 | 6 | 11 | 16 | 21 |
| SNP Position in Target SEQ: nt | 47 | 79 | 51 | 147 | 94 |
| Forward Primer: SEQ ID NO. | 2 | 7 | 12 | 17 | 22 |
| Reverse Primer: SEQ ID NO. | 5 | 10 | 15 | 20 | 25 |
| Probe (Resistant): SEQ ID NO. | 3 | 8 | 13 | 18 | 23 |
| Probe (Susceptible): SEQ ID NO. | 4 | 9 | 14 | 19 | 24 |

As a matter of example, SNP marker 1 (ST2752) at position 82,311,506 bp on chromosome 1 (based on Heinz 1706 SL4.0 assembly) is characterized by a particular sequence polymorphism (resistant donor allele vs. susceptible allele) at position 47 of the target sequence of SEQ ID NO: 1.

### Example 6

### Assessment of Resistance Against other Tobamoviruses

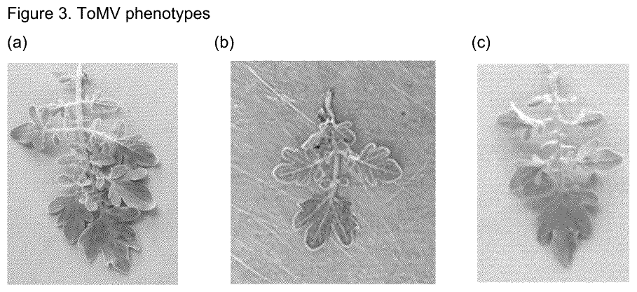

In addition to being screened for ToBRFV resistance, Solanum galapagense accession LA0483 was also challenged against other Tobamoviruses such as TMV and ToMV pathotypes 0 and 2. It was found that this accession provides strong resistance against, e.g., ToMV pathotypes 0 (aucuba) and 2. It is hypothesized that QTL1 of the invention is not only responsible for the ToBRFV resistance but also for a broad-spectrum resistance against Tobamoviruses. The phenotyping results are summarized in Table 5 below as well as in FIG. 3.

TABLE 5
ToBRFV and ToMV phenotypes of original
donor and susceptible check.

| | | ToMV | ToMV |
|---|---|---|---|
| | ToBRFV | pathotype 0 | pathotype 2 |
| Material No, ID and type | score | (aucuba) score | score |
| 1. LA0483: Original donor | HR | HR | HR |
| 2. 16TEP070330: Susceptible | S | S | S |
| parent | | | |

### BIBLIOGRAPHY

Luria et al., 2017, A new Israeli Tobamovirus isolate infects tomato plants harboring Tm-22 resistance genes, PLOS ONE 12 (1): e0170429. doi:10.1371/journal.pone.0170429
Salem et al., 2016, A new tobamovirus infecting tomato crops in Jordan, Arch. Virol. 161:503-506.
Zinger et al., 2021, Identification and mapping of tomato genome loci controlling tolerance and resistance to Tomato Brown Rugose Fruit Virus, Plants 10(1):179. doi: 10.3390/plants10010179.

1. A cultivated tomato plant, preferably a cultivated Solanum lycopersicum plant resistant to ToBRFV infection, comprising in its genome an introgressed sequence from Solanum galapagense which confers resistance to ToBRFV, wherein said introgressed sequence is located on chromosome 1 and comprises at least one of the following SNP markers:
a) an A genotype in the homozygous state for SNP marker 2 at a position corresponding to position 79 in SEQ ID NO: 6;
b) a G genotype in the homozygous state for SNP marker 3 at a position corresponding to position 51 in SEQ ID NO: 11; and/or
c) a G genotype in the homozygous state for SNP marker 4 at a position corresponding to position 147 in SEQ ID NO: 16.

2. The plant according to claim 1, wherein:
a) the A genotype for SNP marker 2 can be identified in a PCR by amplification of a nucleic acid fragment with a pair of oligonucleotide primers: forward primer of SEQ ID NO: 7 and reverse primer of SEQ ID NO: 10, and probe of SEQ ID NO: 8;
b) the G genotype for SNP marker 3 can be identified in a PCR by amplification of a nucleic acid fragment with a pair of oligonucleotide primers: forward primer of SEQ ID NO: 12 and reverse primer of SEQ ID NO: 15, and probe of SEQ ID NO: 13; and/or
c) the G genotype for SNP marker 4 can be identified in a PCR by amplification of a nucleic acid fragment with a pair of oligonucleotide primers: forward primer of SEQ ID NO: 17 and reverse primer of SEQ ID NO: 20, and probe of SEQ ID NO: 18.

3. The plant according to claim 1, wherein said introgressed sequence comprises at least one of SEQ ID NO: 6, SEQ ID NO: 11, and/or SEQ ID NO: 16, or a sequence that is at least 80% identical to one or more of said sequences.

4. The plant of claim 1, wherein said introgressed sequence is comprised in tomato plant 19TEP080670, representative seed of which is deposited under NCIMB Accession No. 43938, or a progeny or an ancestor thereof.

5. The plant of claim 1 wherein said plant is obtained by crossing tomato plant 19TEP080670, representative seed of which is deposited under NCIMB Accession No. 43938, or a progeny or an ancestor thereof, or Solanum galapagense accession LA0483, with a tomato plant that does not contain said ToBRFV resistance-conferring introgressed sequence.

6. The plant of claim 1 wherein said plant is an inbred, a dihaploid, a diploid, or a hybrid plant.

7. (canceled)

8. A plant part of a plant according to claim 1.

9. A seed that produces a plant or a plant part according to claim 1.

10. A method for producing a cultivated tomato plant, preferably a cultivated Solanum lycopersicum plant resistant to ToBRFV infection comprising the steps of
a) crossing a plant according to claim 1 with a cultivated tomato plant lacking said ToBRFV resistance-conferring introgressed sequence;
b) selecting a progeny plant comprising said introgressed sequence located on chromosome 1 conferring resistance to ToBRFV, said selecting step comprising detecting at least one of the following SNP markers:
i) an A genotype in the homozygous state for SNP marker 2 at a position corresponding to position 79 in SEQ ID NO: 6;
ii) a G genotype in the homozygous state for SNP marker 3 at a position corresponding to position 51 in SEQ ID NO: 11; and/or
iii) a G genotype in the homozygous state for SNP marker 4 at a position corresponding to position 147 in SEQ ID NO: 16;
thereby producing a plant with enhanced resistance to ToBRFV.

11. The method according to claim 10, wherein the method further comprises:
c) selfing the selected progeny or crossing the selected progeny with another tomato plant to produce further progeny.

12. The method according to claim 11, wherein further progeny is selected and selfed/crossed for 2 to 10 more generations.

13. The method according to claim 10 wherein the plant of step a) is tomato plant 19TEP080670, representative seed of which is deposited under NCIMB Accession No. 43938, or a progeny or an ancestor thereof, or Solanum galapagense accession LA0483.

14. A method for producing a F1 tomato plant exhibiting resistance to ToBRFV, the method comprising crossing an inbred tomato plant, which is a plant according to claim 1, with a different inbred tomato plant to produce F1 hybrid progeny.

15. A method for identifying a cultivated tomato plant, preferably a cultivated Solanum lycopersicum plant resistant to ToBRFV infection and having at least one copy of said ToBRFV resistance-conferring introgressed sequence, said method comprising the step of detecting at least one of the following SNP markers:
a) an A genotype in the homozygous state for SNP marker 2 at a position corresponding to position 79 in SEQ ID NO: 6;
b) a G genotype in the homozygous state for SNP marker 3 at a position corresponding to position 51 in SEQ ID NO: 11; and/or
c) a G genotype in the homozygous state for SNP marker 4 at a position corresponding to position 147 in SEQ ID NO: 16;
thereby identifying a tomato plant exhibiting resistance to ToBRFV.

16. The method according to claim 15, wherein said method further comprises selecting a tomato plant comprising said one or more SNP markers and crossing the selected tomato plant with a second tomato plant to produce progeny tomato plants that comprise at least one of said SNP markers and exhibits resistance to ToBRFV.

17. A method of producing tomato seed, the method comprising growing a tomato plant from the seed of claim 9 and allowing the plant to produce further tomato seed.

18. (canceled)

19. A method of identifying in a cultivated tomato plant, preferably a cultivated Solanum lycopersicum plant, an introgressed sequence associated with an increased resistance to ToBRFV, said method comprising the step of detecting in said plant an allele of at least one DNA marker that is genetically linked to a QTL locus associated with said increased resistance to ToBRFV, wherein said allele maps within 10 cM, preferably within 5 cM of said QTL locus located on chromosome 1 in a genomic region flanked by SNP markers 1 and 5.

20. The method according to claim 19, wherein said QTL locus can be identified by at least one of the following SNP markers
a) an A genotype in the homozygous state for SNP marker 2 at a position corresponding to position 79 in SEQ ID NO: 6;
b) a G genotype in the homozygous state for SNP marker 3 at a position corresponding to position 51 in SEQ ID NO: 11; and/or
c) a G genotype in the homozygous state for SNP marker 4 at a position corresponding to position 147 in SEQ ID NO: 16.

21. The method according to claim 20 further comprising the step of selecting a cultivated tomato plant, preferably a cultivated Solanum lycopersicum plant comprising said introgressed sequence.

22. (canceled)

23. A method of identifying a wild tomato source of ToBRFV resistance trait on chromosome 1, comprising:
a) providing a wild tomato accession or a plurality of wild tomato accessions,
b) screening said wild tomato accession or plurality of wild tomato accessions using a kit which detects at least one of the SNP markers listed in Table 4, and,
c) identifying a wild tomato accession comprising said at least one SNP marker selected in the list of Table 4.

24. (canceled)
